(19) United States
(12) Patent Application Publication
(10) Pub. No.: US 2006/0242771 A1
(43) Pub. Date: Nov. 2, 2006

(54) Anhydrous composition in the form of a film comprising a film-forming polymer and a direct dye, preparation and dyeing process using the composition

(76) Inventors: Eric Bone, Tokyo (JP); Jean Cotteret, Verneuil Sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/362,791

(22) Filed: Feb. 28, 2006

Related U.S. Application Data
(60) Provisional application No. 60/681,952, filed on May 18, 2005.

(30) Foreign Application Priority Data
Feb. 28, 2005 (FR) 05 02032

(51) Int. Cl. A61K 8/00

(52) U.S. Cl. 8/405

(57) ABSTRACT

The present disclosure relates to an anhydrous composition in the form of a film comprising at least one film-forming polymer and at least one direct dye, the total amount of direct dye being at least 3% by weight relative to the weight of the composition. The present disclosure also relates to a process for preparing such a composition, in which a precursor composition comprising, in a suitable solvent, a mixture comprising at least one direct dye and at least one film-forming polymer is applied to a support; and the solvent is then evaporated off. Further, the present disclosure relates to a process for dyeing keratin fibers, which consists in placing the fibers and the composition in contact, in the presence of an aqueous medium.

This application claims benefit of U.S. Provisional Application No. 60/681,952, filed May 18, 2005, the contents of which are incorporated herein by reference. This application also claims benefit of priority under 35 U.S.C. §119 to French Patent Application No. FR 05 02032, filed Feb. 28, 2005, the contents of which are also incorporated herein by reference.

The present disclosure relates to an anhydrous composition in the form of a film comprising at least one film-forming polymer and at least one direct dye, and also to a process for preparing the composition. The present disclosure also relates to a process for dyeing keratin fibers, for example human keratin fibers, using such a composition.

It has been known for a long time that the color of the hair may be modified, for example to mask grey hair.

Essentially two types of technology used for dyeing human keratin fibers are known.

The first method, known as direct dyeing or semi-permanent dyeing, comprises changing or providing color by applying a colored molecule that penetrates by diffusion into the fiber and/or that rests absorbed onto its surface. This coloration may also be performed in the presence of an alkaline agent under oxidizing conditions. In such a case, lightening of the fibers accompanied by coloration thereof are simultaneously observed. This is referred to as direct dyeing under lightening conditions.

The second method, known as oxidation dyeing or permanent dyeing, comprises changing or providing color by performing inside the fiber itself an oxidative condensation of dye precursors that are sparingly colored or uncolored compounds. After this reaction, the dyes formed are insoluble and are trapped within the fiber.

Both methods summarized above allow access to numerous strong, relatively fast and sparingly selective colors.

The present disclosure relates to the field of direct dyeing.

One of the drawbacks occasionally encountered lies in the fact that these dyes may be fragile or even unstable in dilute aqueous solution, whether or not the dyes are in the presence of alkaline agents.

In addition, many direct dyes are unstable in the presence of reducing or oxidizing agents.

To overcome these drawbacks, it has been proposed to present them in specific packaging, for instance sachets for powders, or to combine them in the composition with efficient stabilizers.

Other difficulties may also be encountered due to the fact that, very frequently, the dye compositions are mixed extemporaneously, before application, with additional compositions. Thus, such embodiments lead to long and occasionally difficult implementations while at the same time not ensuring total homogeneity of the composition to be applied to the fibers.

The present disclosure seeks to solve at least one of the drawbacks mentioned above.

At least one aspect of the present disclosure relates to a composition in the form of a film comprising at least one film-forming polymer and at least one direct dye, the total amount of direct dye being at least 3% by weight relative to the weight of the composition.

Another aspect of the present disclosure relates to a process for preparing such a composition, in which a precursor composition comprising, in a suitable solvent, a mixture comprising at least one direct dye and at least one film-forming polymer, is applied to a support; and the solvent is then evaporated off.

Another aspect of the present disclosure relates to a process for dyeing keratin fibers, such as human keratin fibers, which comprises placing the fibers and the presently disclosed composition in contact, in the presence of an aqueous medium.

The presently disclosed composition makes it possible to solve the problem of stability of direct dyes during storage, without necessarily using stabilizers. The reason for this is that the direct dyes are protected by the polymer film.

It should moreover be noted that the conditions for applying the composition are simplified. For instance, the step of mixing the dye composition and the oxidizing composition before application to the fibers may be dispensed with. Such mixing may also be performed, but the nature of the composition as disclosed herein is such that the mixing and homogenization of the compositions are considerably simplified.

Other characteristics of the present disclosure will emerge more clearly on reading the description and the examples that follow.

In the text herein below, and unless otherwise indicated, when a range of values is provided, the limits of that range are included in the range thus defined.

The present disclosure applies to the treatment of keratin fibers, for instance human keratin fibers such as the hair.

For the purposes of the present disclosure, the composition is considered as being anhydrous since its water amount is less than 10% by weight of the composition, such as less than 5% by weight relative to the weight of the composition, and further for example less than 3% by weight relative to the weight of the composition. In at least one embodiment, the composition contains no water.

As indicated above, the anhydrous composition disclosed herein may be in the form of a film comprising at least one film-forming polymer and at least one direct dye, the total amount of direct dye being at least 3% by weight relative to the weight of the composition.

The direct dye(s) included in the composition according to the present disclosure may be chosen from the direct dyes conventionally used in the field of dyeing keratin fibers, for instance human keratin fibers.

Thus, these direct dyes may be ionic or nonionic species. For example, the direct dyes may be chosen from cationic and nonionic species.

Non-limiting examples that may be mentioned include nitrobenzene dyes, azo dyes, azomethine dyes, methine dyes, tetraazapentamethine dyes, anthraquinone dyes, naphthoquinone dyes, benzoquinone dyes, phenothiazine dyes, indigoid dyes, xanthene dyes, phenanthridine dyes, phthalocyanin dyes, triarylmethane-based dyes and natural dyes, and mixtures thereof.

For example, the direct dyes may be chosen from the following red or orange nitrobenzene dyes:
1-hydroxy-3-nitro-4-N-(γ-hydroxypropyl)aminobenzene,
N-(β-hydroxyethyl)amino-3-nitro-4-aminobenzene,
1-amino-3-methyl-4-N-(β-hydroxyethyl)amino-6-nitrobenzene,
1-hydroxy-3-nitro-4-N-(β-hydroxyethyl)aminobenzene,
1,4-diamino-2-nitrobenzene,
1-amino-2-nitro-4-methylaminobenzene,
N-(β-hydroxyethyl)-2-nitro-para-phenylenediamine,
1-amino-2-nitro-4-(β-hydroxyethyl)amino-5-chlorobenzene,
2-nitro-4-aminodiphenylamine,
1-amino-3-nitro-6-hydroxybenzene,
1-(β-aminoethyl)amino-2-nitro-4-(β-hydroxyethyloxy)benzene,
1-(β,γ-dihydroxypropyl)oxy-3-nitro-4-(β-hydroxyethyl)aminobenzene,
1-hydroxy-3-nitro-4-aminobenzene,
1-hydroxy-2-amino-4,6-dinitrobenzene,
1-methoxy-3-nitro-4-(β-hydroxyethyl)aminobenzene,
2-nitro-4′-hydroxydiphenylamine, and
1-amino-2-nitro-4-hydroxy-5-methylbenzene.

The direct dye may also be chosen from yellow and green-yellow nitrobenzene direct dyes; non-limiting mention may be made, for example, of the compounds chosen from:
1-β-hydroxyethyloxy-3-methylamino-4-nitrobenzene,
1-methylamino-2-nitro-5-(β,γ-dihydroxypropyl)oxybenzene,
1-(β-hydroxyethyl)amino-2-methoxy-4-nitrobenzene,
1-(β-aminoethyl)amino-2-nitro-5-methoxybenzene,
1,3-di(β-hydroxyethyl)amino-4-nitro-6-chlorobenzene,
1-amino-2-nitro-6-methylbenzene,
1-(β-hydroxyethyl)amino-2-hydroxy-4-nitrobenzene,
N-(β-hydroxyethyl)-2-nitro-4-trifluoromethylaniline,
4-(β-hydroxyethyl)amino-3-nitrobenzenesulfonic acid,
4-ethylamino-3-nitrobenzoic acid,
4-(β-hydroxyethyl)amino-3-nitrochlorobenzene,
4-(β-hydroxyethyl)amino-3-nitromethylbenzene,
4-(β,γ-dihydroxypropyl)amino-3-nitrotrifluoromethylbenzene,
1-(β-ureidoethyl)amino-4-nitrobenzene,
1,3-diamino-4-nitrobenzene,
1-hydroxy-2-amino-5-nitrobenzene,
1-amino-2-[tris(hydroxymethyl)methyl]amino-5-nitrobenzene,
1-(β-hydroxyethyl)amino-2-nitrobenzene, and
4-(β-hydroxyethyl)amino-3-nitrobenzamide.

Non-limiting mention may also be made of blue or violet nitrobenzene direct dyes, for instance:
1-(β-hydroxyethyl)amino-4-N, N-bis(β-hydroxyethyl)amino-2-nitrobenzene,
1-(γ-hydroxypropyl)amino-4,N,N-bis(β-hydroxyethyl)amino-2-nitrobenzene,
1-(γ-hydroxyethyl)amino-4-(N-methyl-N-β-hydroxyethyl)amino-2-nitrobenzene,
1-(β-hydroxyethyl)amino-4-(N-ethyl-N-β-hydroxyethyl)amino-2-nitrobenzene,
1-(β,γ-dihydroxypropyl)amino-4-(N-ethyl-N-β-hydroxyethyl)amino-2-nitrobenzene, and
2-nitro-para-phenylenediamines having the following formula: 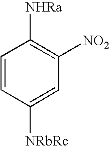
   in which:
R_{b} is chosen from C₁-C₄ alkyl, β-hydroxyethyl, β-hydroxypropyl and γ-hydroxypropyl radicals;
Rₐ and R_{c}, which may be identical or different, are chosen from β-hydroxyethyl, β-hydroxypropyl, γ-hydroxypropyl and β,γ-dihydroxypropyl radicals, wherein at least one of the radicals R_{b}, R_{c} or Rₐ is a γ-hydroxypropyl radical and R_{b} and R_{c} do not simultaneously denote a β-hydroxyethyl radical when R_{b} is a γ-hydroxypropyl radical, such as those described in French Patent No. FR 2 692 572.

Among the azo direct dyes that may be used according to the present disclosure, non-limiting mention may be made of the cationic azo dyes described in patent applications WO 95/15144, WO 95/01772 and EP 714 954, FR 2 822 696, FR 2 825 702, FR 2 825 625, FR 2 822 698, FR 2 822 693, FR 2 822 694, FR 2 829 926, FR 2 807 650, WO 02/078660, WO 02/100834, WO 02/100369 and FR 2 844 269.

Among these compounds, mention may be made but is not limited to the following dyes:
1,3-dimethyl-2-[[4-(dimethylamino)phenyl]azo]-1 H-imidazolium chloride,
1,3-dimethyl-2-[(4-aminophenyl)azo]-1H-imidazolium chloride, and
1-methyl-4-[(methylphenylhydrazono)methyl]pyridinium methyl sulfate.

Among the azo direct dyes, mention may also be made of the following dyes, described in the Colour Index International 3rd edition: Disperse Red 17, Acid Yellow 9, Acid Black 1, Basic Red 22, Basic Red 76, Basic Yellow 57, Basic Brown 16, Acid Yellow 36, Acid Orange 7, Acid Red 33, Acid Red 35, Basic Brown 17, Acid Yellow 23, Acid Orange 24, and Disperse Black 9.

Non-limiting mention may also be made of 1-(4′-aminodiphenylazo)-2-methyl-4-bis(β-hydroxyethyl)aminobenzene, and 4-hydroxy-3-(2-methoxyphenylazo)-1-naphthalenesulfonic acid.

The quinone direct dyes that may be mentioned include but are not limited to the following dyes: Disperse Red 15, Solvent Violet 13, Acid Violet 43, Disperse Violet 1, Disperse Violet 4, Disperse Blue 1, Disperse Violet 8, Disperse Blue 3, Disperse Red 11, Acid Blue 62, Disperse Blue 7, Basic Blue 22, Disperse Violet 15, Basic Blue 99, and also the following compounds:
1-N-methylmorpholiniumpropylamino-4-hydroxyanthraquinone
1-aminopropylamino-4-methylaminoanthraquinone
1-aminopropylaminoanthraquinone
5-β-hydroxyethyl-1,4-diaminoanthraquinone
2-aminoethylaminoanthraquinone, and
1,4-bis(β,γ-dihydroxypropylamino)anthraquinone.

The azine dyes that may be mentioned include but are not limited to the following compounds:
Basic Blue 17 and Basic Red 2.

The cationic methine direct dyes that may also be mentioned include but are not limited to Basic Red 14, Basic Yellow 13 and Basic Yellow 29.

The triarylmethane dyes that may be used according to the present disclosure, non-limiting mention may be made of the following compounds: Basic Green 1, Acid Blue 9, Basic Violet 3, Basic Violet 14, Basic Blue 7, Acid Violet 49, Basic Blue 26, and Acid Blue 7.

The indoamine dyes that may be used according to the present disclosure, non-limiting mention may be made of the following compounds:
2-β-hydroxyethylamino-5-[bis(β-4′-hydroxyethyl)amino]anilino-1,4-benzoquinone;
2-β-hydroxyethylamino-5-(2′-methoxy-4′-amino)anilino-1,4-benzoquinone;
3-N(2′-chloro-4′-hydroxy)phenylacetylamino-6-methoxy-1,4-benzoquinoneimine;
3-N(3′-chloro-4′-methylamino)phenylureido-6-methyl-1,4-benzoquinoneimine;
3-[4′-N-(ethylcarbamylmethyl)amino]phenylureido-6-methyl-1,4-benzoquinoneimine.

The composition may also comprise natural direct dyes, for instance lawsone, juglone, alizarin, purpurin, carminic acid, kermesic acid, purpurogallin, protocatechaldehyde, indigo, isatin, curcumin, spinulosin and apigenidin.

According to at least one embodiment disclosed herein, the total amount of direct dye ranges from 3% to 99.5% by weight relative to the weight of the composition.

For instance, the total amount of direct dye ranges from 10% to 80% by weight relative to the weight of the composition.

The composition according to the present disclosure also comprises at least one film-forming polymer, which may be nonionic, cationic, anionic or amphoteric.

It should be noted that the film-forming polymer included in the composition as disclosed herein is such that, under the conditions of application of the composition to the keratin fibers, i.e., in the presence of an aqueous medium and/or massaging of the fibers and/or of homogenization, the composition loses its cohesion and disintegrates.

For instance, the film-forming polymer is chosen from vinylpyrrolidone-based polymers, polyvinyl alcohol, polyurethanes, polymers derived from caprolactam, vinyllactam or vinyl acetate, acrylamide-based polymers, polysaccharides capable of forming a film in dry form, such as cellulose derivatives, starches and derivatives, pullulan gum, gum arabic, pectins, alginates, carrageenans, galactomannans, agars, chitosans, chitins, polymers derived from hyaluronic acid, xanthan gum, karaya gum, proteins capable of forming a film in dry form such as gelatin, gluten, casein, zein, gliadin, hordein and natural or synthetic derivatives thereof, silicone-based polymers, amphoteric or anionic polymers derived from monomers comprising at least one carboxylic, sulfonic or phosphoric function, acrylic copolymers of phosphorylcholine (lipidure), and anion-cation complexes such as gum arabic/gelatin or gum arabic/chitosan, or the collagen/glycosaminoglycan combination.

As suitable cationic film-forming polymers, non-limiting mention may be made of the following polymers, generally having a number-average molecular mass ranging from 500 to 5 000 000:
(1) homopolymers or copolymers derived from acrylic or methacrylic esters or amides and containing at least one of the units of the following formulae: 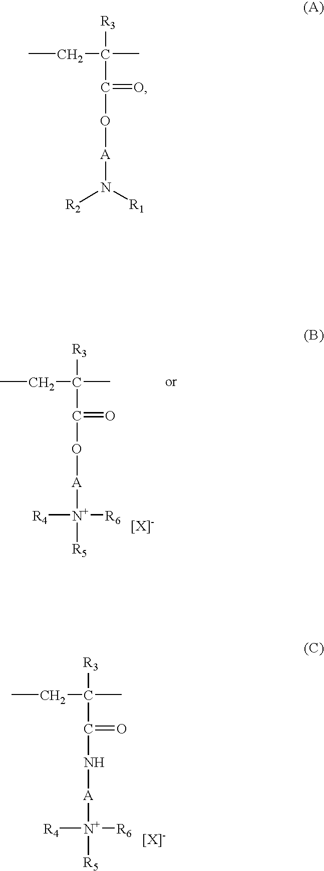
   in which:

R₁ and R₂, which may be identical or different, each are chosen from a hydrogen atom and alkyl groups having from 1 to 6 carbon atoms;

R₃ is chosen from a hydrogen atom and a CH₃ group;

A is chosen from linear or branched alkyl groups containing from 1 to 6 carbon atoms and hydroxyalkyl groups containing from 1 to 4 carbon atoms;

R₄, R₅ and R₆, which may be identical or different, are chosen from alkyl groups having from 1 to 18 carbon atoms and from benzyl groups;

X is chosen from a methosulfate anion and a halide ion such as chloride or bromide.
(2) quaternized guar gums;
(3) quaternary copolymers of vinylpyrrolidone and of vinylimidazole;
(4) chitosans or salts thereof;
   the salts that may be used include but are not limited to chitosan acetate, lactate, glutamate, gluconate, and pyrrolidonecarboxylate.

The copolymers of the family (1) also contain one or more units derived from comonomers that can be chosen from the family of acrylamides, methacrylamides, diacetone acrylamides, acrylamides and methacrylamides substituted on the nitrogen with (C₁₋₄) lower alkyl groups, groups derived from acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulfate or with a dimethyl halide,
copolymers of acrylamide and of methacryloyloxyethyltrimethylammonium chloride, described, for example, in patent application EP-A-080 976,
copolymers of acrylamide and of methacryloyloxyethyltrimethylammonium methosulfate,
quaternized or nonquaternized vinylpyrrolidone/dialkylaminoalkyl acrylate and methacrylate copolymers, such as the products sold under the name “Gafquat®” by the company ISP, for instance “Gafquat® 734” or “Gafquat® 755” and alternatively the products known as “Copolymer® 845, 958 and 937”. These polymers are described in detail in French Patent Nos. FR 2 077 143 and FR 2 393 573,
dimethylaminoethyl methacrylate/vinylcaprolactam/vinylpyrrolidone terpolymers, such as the product sold under the name Gaffix® VC 713 by the company ISP, and
quaternized vinylpyrrolidone/dimethylaminopropyl methacrylamide copolymers, such as the product sold under the name “Gafquat® HS 100” by the company ISP.

Among these compounds, non-limiting mention may be made of chitosan having a degree of deacetylation of 90% by weight, and the chitosan pyrrolidonecarboxylate sold under the name Kytamer® PC by the company Amerchol.

As regards the anionic film-forming polymers, they generally comprise at least one group derived from carboxylic acid, sulfonic acid or phosphoric acid and have a number-average molecular mass ranging from 500 to 5 000 000.

The carboxylic groups may be chosen from unsaturated monocarboxylic or dicarboxylic acid monomers such as those corresponding to the formula: 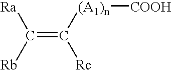
in which n is an integer from 0 to 10, A₁ is a methylene group, optionally connected to the carbon atom of the unsaturated group, or to the neighbouring methylene group when n is greater than 1, via a hetero atom such as oxygen or sulfur, Rₐ is chosen from a hydrogen atom, a phenyl group, and a benzyl group, R_{b} is chosen from a hydrogen atom and a (C₁₋₄)alkyl group, such as methyl, ethyl or carboxyl, R_{c} is chosen from a hydrogen atom, a lower alkyl group, a —CH₂—COOH group, a phenyl group, and a benzyl group.

The anionic film-forming polymers containing carboxylic groups that may be used include but are not limited to:
A) acrylic or methacrylic acid homo- or copolymers, or salts thereof, for example the products sold under the names Versicol® E or K by the company Allied Colloid and Ultrahold® by the company BASF, copolymers of acrylic acid and of acrylamide, and sodium salts of polyhydroxycarboxylic acid;
B) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described in French Patent No. FR 1 222 944 and German Patent Application No. 2 330 956, the copolymers of this type containing an optionally N-alkylated and/or hydroxyalkylated acrylamide unit in their chain as described in Luxembourg Patent Applications Nos. 75370 and 75371. Mention may also be made of copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by the company ISP under the name Acrylidone® LM and methacrylic acid/ethyl acrylate/tert-butyl acrylate terpolymers such as the product sold under the name Luvimer® 100 P by the company BASF;
C) copolymers derived from crotonic acid such as those containing vinyl acetate or propionate units in their chain and optionally other monomers such as allylic esters or methallylic esters, vinyl ether or vinyl ester of a linear or branched saturated carboxylic acid with a long hydrocarbon-based chain such as those containing at least 5 carbon atoms, it being possible for these polymers optionally to be grafted and crosslinked, or alternatively another vinyl, allylic or methallylic ester monomer of an α- or β-cyclic carboxylic acid. Such polymers are described, inter alia, in French Patents Nos. FR 1 222 944, FR 1 580 545, FR 2 265 782, FR 2 265 781, FR 1 564 110 and FR 2 439 798. A commercial product falling into this class is the resin 28-29-30 sold by the company National Starch;
D) copolymers derived from C₄-C₈ mono-unsaturated carboxylic acids or anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described in U.S. Pat. No. 2,047,398; U.S. Pat. No. 2,723,248 and U.S. Pat. No. 2,102,113 and GB Patent No. 839 805. Commercial products may be sold under the names Gantrez® AN or ES by the company ISP,
copolymers comprising (i) one or more maleic, citraconic or itaconic anhydride units and (ii) one or more monomers chosen from allylic or methallylic esters optionally containing one or more acrylamide, methacrylamide or α-olefin groups, acrylic or methacrylic esters, acrylic or methacrylic acids or vinylpyrrolidone in their chain, the anhydride functions of these copolymers optionally being monoesterified or monoamidated.

These polymers are described, for example, in French Patent Nos. FR 2 350 384 and FR 2 357 241;
E) polyacrylamides containing carboxylate groups;
F) anionic polyurethanes, such as the product sold by BASF under the name Luviset PUR.

The polymers comprising sulfonic groups are polymers containing vinylsulfonic, styrenesulfonic, naphthalenesulfonic or acrylamidoalkylsulfonic units.

These polymers may be chosen from:
polyvinylsulfonic acid salts having a molecular weight ranging from 1000 to 100 000, as well as the copolymers with an unsaturated comonomer such as acrylic or methacrylic acids and their esters, as well as acrylamide or its derivatives, vinyl ethers and vinylpyrrolidone;
polystyrenesulfonic acid salts, such as the sodium salts sold for example under the name Flexan® 130 by National Starch. These compounds are described in patent FR 2 198 719;
polyacrylamidesulfonic acid salts, such as those mentioned in U.S. Pat. No. 4,128,631 such as, for example polyacrylamidoethylpropanesulfonic acid.

According to the present disclosure, it is also possible to use film-forming anionic polymers of grafted silicone type comprising a polysiloxane portion and a portion comprising a non-silicone organic chain, one of the two portions constituting the main chain of the polymer, the other being grafted onto the said main chain. These polymers are described, for example, in patent applications EP-A-0 412 704, EP-A-0 412 707, EP-A-0 640 105 and WO 95/00578, EP-A-0 582 152 and WO 93/23009 and U.S. Pat. No. 4,693,935; U.S. Pat. No. 4,728,571 and U.S. Pat. No. 4,972,037.

Such polymers are, for example, copolymers that can be obtained by radical polymerization from the monomer mixture comprising:
a) 50 to 90% by weight of tert-butyl acrylate;
b) 0 to 40% by weight of acrylic acid;
c) 5 to 40% by weight of silicone macromer of formula: 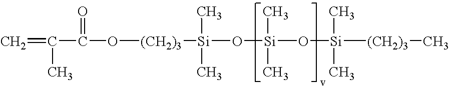
   with v being a number from 5 to 700; the weight percentages being calculated relative to the total weight of the monomers.

Other examples of grafted silicone polymers are, for instance, polydimethylsiloxanes (PDMSs) onto which are grafted, via a thiopropylene-type connecting chain, mixed polymer units of the poly(meth)acrylic acid type and of the polyalkyl(meth)acrylate type and polydimethylsiloxanes (PDMSs) onto which are grafted, via a thiopropylene-type connecting chain, polymer units of the polyisobutyl(meth)acrylate type.

Functionalized silicone or non-silicone polyurethanes may also be used as film-forming polymers.

The polyurethanes useful in the present disclosure include but are not limited to those disclosed in patents EP-A-0 751 162, EP-A-0 637 600, FR-A-2 743 297 and EP-A-0 648 485, and patents EP-A-0 656 021 or WO 94/03510 and EP-A-0 619 111.

According to the present disclosure, the anionic film-forming polymers may be chosen from acrylic acid copolymers, such as the acrylic acid/ethyl acrylate/N-tert-butylacrylamide terpolymers sold under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold for example under the name Gantrez® by the company ISP, the copolymers of methacrylic acid and of methyl methacrylate sold under the name Eudragit® L by the company Rohm Pharma, the copolymers of methacrylic acid and of ethyl acrylate sold under the name Luvimer® MAEX or MAE by the company BASF and the vinyl acetate/crotonic acid copolymers and the vinyl acetate/crotonic acid copolymers grafted with polyethylene glycol sold under the name Aristoflex® A by the company BASF, and also the polyurethane Luviset PUR® sold by the company BASF.

In at least one embodiment, the anionic film-forming polymers may be chosen from the methyl vinyl ether/monoesterified maleic anhydride copolymers sold under the name Gantrez® ES 425 by the company ISP, the acrylic acid/ethyl acrylate/N-tert-butylacrylamide terpolymers sold under the name Ultrahold® Strong by the company BASF, the copolymers of methacrylic acid and of methyl methacrylate sold under the name Eudragit® L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer® MAEX or MAE by the company BASF and the vinylpyrrolidone/acrylic acid/lauryl methacrylate terpolymers sold under the name Acrylidone® LM by the company ISP, and also the polyurethane Luviset PUR® sold by the company BASF.

Among the amphoteric film-forming polymers that may be used in accordance with the present disclosure, non-limiting mention may be made of those comprising units B and C distributed randomly in the polymer chain, in which B is a unit derived from a monomer containing at least one basic nitrogen atom and C is a unit derived from an acid monomer containing one or more carboxylic or sulfonic groups, or alternatively B and C may denote groups derived from carboxybetaine or sulfobetaine zwitterionic monomers;

B and C may also denote a cationic polymer chain containing primary, secondary, tertiary or quaternary amine groups, in which at least one of the amine groups bears a carboxylic or sulfonic group connected via a hydrocarbon-based group or alternatively B and C form part of a chain of a polymer containing an a-dicarboxylic ethylene unit in which one of the carboxylic groups has been made to react with a polyamine containing one or more primary or secondary amine groups.

The amphoteric film-forming polymers corresponding to the definition disclosed herein may be chosen from the following polymers:
(1) polymers resulting from the copolymerization of a monomer derived from a vinyl compound bearing a carboxylic group such as acrylic acid, methacrylic acid, maleic acid, α-chloroacrylic acid, and a basic monomer derived from a substituted vinyl compound containing at least one basic atom (for example an amino function), such as dialkylaminoalkyl methacrylate and acrylate, dialkylaminoalkylmethacrylamides and -acrylamides. Such compounds are described in U.S. Pat. No. 3,836,537. Mention may also be made of the sodium acrylate/acrylamidopropyltrimethylammonium chloride copolymer sold under the name Polyquart KE 3033 by the company Henkel.

The vinyl compound may also be a dialkyldiallylammonium salt such as diethyidiallylammonium chloride. The copolymers of acrylic acid and of the latter monomer are sold under the names Merquat 280, Merquat 295 and Merquat Plus 3330 by the company Calgon.
(2) polymers containing units derived from:

a) at least one monomer chosen from acrylamides and methacrylamides substituted on the nitrogen atom with an alkyl group,

b) at least one acidic co-monomer containing one or more reactive carboxylic groups, and

c) at least one basic co-monomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides that may be used according to the present disclosure are the compounds in which the alkyl groups contain from 2 to 12 carbon atoms such as, for example N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers may be chosen from acrylic acid, methacrylic acid, crotonic acid, itaconic acid, maleic acid and fumaric acid and alkyl monoesters, having 1 to 4 carbon atoms, of maleic or fumaric acids or anhydrides.

The basic comonomers include but are not limited to aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold under the name Amphomer® or Lovocryl® 47 by the company National Starch, may also be used.
(3) crosslinked and acylated polyamino amides partially or totally derived from polyamino amides of general formula: 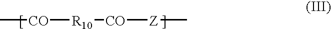
   in which R₁₀ is a divalent group derived from a saturated dicarboxylic acid, a mono- or dicarboxylic aliphatic acid containing an ethylenic double bond, an ester of a lower alkanol, having 1 to 6 carbon atoms, of these acids, or a group derived from the addition of any one of the said acids to a bis(primary) or bis(secondary)amine, and Z is a group derived from a bis(primary), mono- or bis(secondary)polyalkylene-polyamine and may represent:

a) in proportions of from 60 to 100 mol %, the group

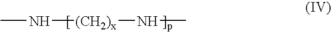

where x=2 and p=2 or 3, or alternatively x=3 and p=2

this group being derived from diethylenetriamine, from triethylenetetraamine or from dipropylenetriamine;

b) in proportions of from 0 to 40 mol %, the group (IV) above in which x=2 and p=1 and which is derived from ethylenediamine, or the group derived from piperazine:

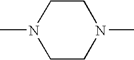

c) in proportions of from 0 to 20 mol %, the —NH—(CH₂)₆—NH— group derived from hexamethylenediamine, these polyamino amides being crosslinked by reaction addition of a difunctional crosslinking agent chosen from epihalohydrins, diepoxides, dianhydrides and bis-unsaturated derivatives, using from 0.025 to 0.35 mol of crosslinking agent per amine group of the polyamino amide and acylated by the action of acrylic acid, chloroacetic acid or an alkane sultone, or salts thereof.

The saturated carboxylic acids may be chosen from acids having 6 to 10 carbon atoms, such as adipic acid, 2,2,4-trimethyladipic acid and 2,4,4-trimethyladipic acid, terephthalic acid, acids containing an ethylenic double bond, for instance acrylic acid, methacrylic acid and itaconic acid.

The alkane sultones used in the acylation include but are not limited to propane sultone or butane sultone, the salts of the acylating agents may be sodium or potassium salts.
(4) polymers containing zwitterionic units of formula: 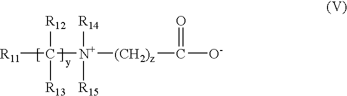
   in which R₁₁ is a polymerizable unsaturated group such as an acrylate, methacrylate, acrylamide or methacrylamide group, y and z represent an integer from 1 to 3, R₁₂ and R₁₃ are independently chosen from a hydrogen atom, and methyl, ethyl and propyl groups, R₁₄ and R₁₅ are independently chosen from a hydrogen atom and an alkyl group such that the sum of the carbon atoms in R₁₄ and R₁₅ does not exceed 10.

The polymers comprising such units may also contain units derived from nonzwitterionic monomers such as dimethyl or diethylaminoethyl acrylate or methacrylate or alkyl acrylates or methacrylates, acrylamides or methacrylamides or vinyl acetate.

By way of example, non-limiting mention may be made of butyl methacrylate/N, N-dimethylcarboxyaminoethyl methacrylate copolymers.
(5) polymers derived from chitosan containing monomer units corresponding to the following formulae: 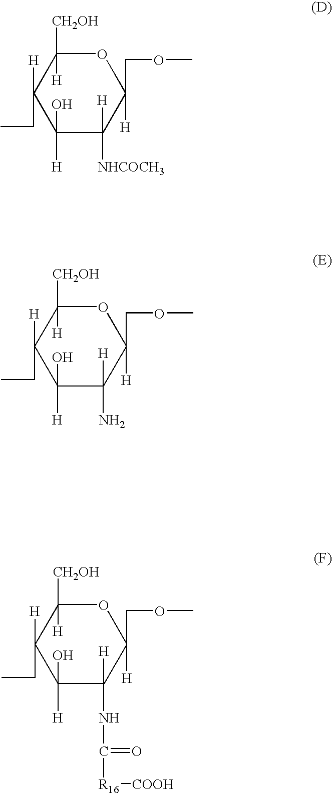
   the unit (D) being present in proportions ranging from 0% to 30%, the unit (E) in proportions ranging from 5% to 50% and the unit (F) in proportions ranging from 30% to 90%, it being understood that, in this unit (F), R₁₆ is a group of formula: 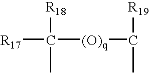
   in which, if q=0, R₁₇, R₁₈ and R₁₉, which may be identical or different, are each chosen from a hydrogen atom, from methyl, hydroxyl, acetoxy, and amino residues, and from monoalkylamine and dialkylamine residues that are optionally interrupted by one or more nitrogen atoms and/or optionally substituted with one or more amine, hydroxyl, carboxyl, alkylthio or sulfonic groups, an alkylthio residue in which the alkyl group bears an amino residue, at least one of the groups R₁₇, R₁₈ and R₁₉ being, in this case, a hydrogen atom; or, if q=1, R₁₇, R₁₈ and R₁₉ are each a hydrogen atom, as well as the salts formed by these compounds with bases or acids.
(6) polymers corresponding to the general formula (VI) are described, for example, in French Patent No. FR 1 400 366 and comprising the repeating unit below: 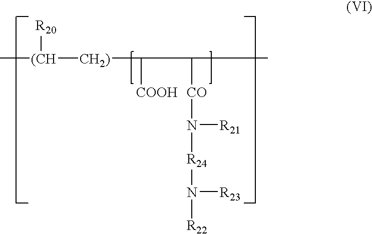
   in which
R₂₀ is chosen from a hydrogen atom and CH₃O, CH₃CH₂O and phenyl groups,
R₂₁ is chosen from a hydrogen atom and lower alkyl groups such as methyl or ethyl,
R₂₂ is chosen from a hydrogen atom and C₁-C₆ lower alkyl groups such as methyl or ethyl,
R₂₄ is chosen from —CH₂—CH₂—, —CH₂—CH₂—CH₂— and —CH₂—CH(CH₃)— groups,
R₂₃ is chosen from C₁-C₆ lower alkyl groups such as methyl or ethyl and groups corresponding to the formula: —R₂₄—N(R₂₂)₂, wherein R₂₄ is a —CH₂—CH₂—, —CH₂—CH₂—CH₂— or —CH₂—CH(CH₃)— group and R₂₂ has the meanings mentioned above.
(7) polymers derived from the N-carboxyalkylation of chitosan, such as N-carboxymethylchitosan or N-carboxybutylchitosan.
(8) amphoteric polymers of the type -D-X-D-X chosen from:

a) polymers obtained by the action of chloroacetic acid or sodium chloroacetate on compounds containing at least one unit of formula: -D-X-D-X-D- (VIl) where D is a group 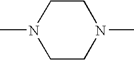
and X denotes the symbol E or E′, E or E′, which may be identical or different, is a divalent group that is an alkylene group containing a straight or branched chain containing up to 7 carbon atoms in the main chain, which is unsubstituted or substituted with hydroxyl groups and which can contain, in addition to oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups.

b) polymers of formula: -D-X-D-X- (VII′)
where D is a group 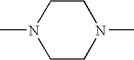
and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interrupted by an oxygen atom and necessarily containing one or more carboxyl functions or one or more hydroxyl functions and betainized by reaction with chloroacetic acid or sodium chloroacetate.
(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkynol. These copolymers may also contain other vinyl comonomers such as vinylcaprolactam.

The amphoteric film-forming polymers that may be used include but are not limited to those of the family (3), such as the copolymers whose CTFA name is octylacrylamide/acrylates/butylaminoethyl methacrylate copolymer, such as the products sold under the names Amphomer®, Amphomer® LV 71 or Lovocryl® 47 by the company National Starch and those of the family (4) such as butyl methacrylate/N,N-dimethylcarboxyaminoethyl methacrylate copolymers.

The nonionic film-forming polymers that may be used according to the present disclosure are chosen, for example, from:
vinyl acetate homopolymers;
copolymers of vinyl acetate and of acrylic ester;
copolymers of vinyl acetate and of ethylene;
copolymers of vinyl acetate and of maleic ester, for example of dibutyl maleate;
copolymers of acrylic esters, for instance copolymers of alkyl acrylates and of alkyl methacrylates, such as the products sold by the company Rohm & Haas under the names Primal® AC-261 K and Eudragit® NE 30 D, by the company BASF under the name 8845, or by the company Hoechst under the name Appretan® N9212;
copolymers of acrylonitrile and of a nonionic monomer chosen, for example, from butadiene and alkyl(meth)acrylates; mention may be made of the products sold under the name CJ 0601 B by the company Rohm & Haas;
styrene homopolymers;
copolymers of styrene and of an alkyl (meth)acrylate, such as the products Mowilith® LDM 6911, Mowilith® DM 611 and Mowilith® LDM 6070 sold by the company Hoechst, and the products Rhodopas® SD 215 and Rhodopas® DS 910 sold by the company Rhodia Chimie;
copolymers of styrene, of alkyl methacrylate and of alkyl acrylate;
nonionic polyurethanes;
copolymers of styrene and of butadiene;
copolymers of styrene, of butadiene and of vinylpyridine;
copolymers of alkyl acrylate and of urethane;
polyamides; and
vinyllactam homopolymers and copolymers.

The alkyl groups in the nonionic polymers mentioned above may have from 1 to 6 carbon atoms.

According to the present disclosure, the film-forming polymers may be nonionic polymers, such as nonionic polymers containing vinyllactam units. They are described in U.S. Pat. No. 3,770,683; U.S. Pat. No. 3,929,735; U.S. Pat. No. 4,521,504; U.S. Pat. No. 5,158,762; and U.S. Pat. No. 5,506,315 and in Patent Applications Nos. WO 94/121148, WO 96/06592 and WO 96/10593. They may be in pulverulent form or in the form of a solution or suspension.

The homopolymers or copolymers containing vinyllactam units comprise units of formula: 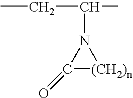
in which n is independently 3, 4 or 5.

The number-average molar mass of the polymers containing vinyllactam units is generally greater than about 5 000, for example from 10 000 to 1 000 000 for instance, such as from 10 000 to 100 000.

Film-forming polymers that may be used in the present disclosure include but are not limited to polyvinylpyrrolidones such as those sold under the name Luviskol® K30 by the company BASF; polyvinylcaprolactams such as those sold under the name Luviskol® PLUS by the company BASF; poly(vinylpyrrolidone/vinyl acetate)copolymers such as those sold under the name PVPVAe S630L by the company ISP, Luviskol® VA 73, VA 64, VA 55, VA 37 and VA 28 by the company BASF; and poly(vinylpyrrolidone/vinyl acetate/vinyl propionate)terpolymers, for instance those sold under the name Luviskol® VAP 343 by the company BASF.

The film-forming polymers included in the composition as disclosed herein may be chosen from soluble or water-dispersible film-forming polymers. The film-forming polymers may be chosen from water-soluble film-forming polymers.

The film-forming polymer may be present in the composition in an amount ranging from 0.5% to 97% by weight relative to the weight of the composition, such as from 5% to 90% by weight relative to the weight of the composition.

The composition according to the present disclosure may also comprise at least one plasticizer.

The plasticizers, when they are present, are chosen from the compounds conventionally used in this field.

For example, the plasticizer is chosen from glycerol, sorbitol, monosaccharides and/or disaccharides, dipropylene glycol, butylene glycol, pentylene glycol or polyethylene glycol, for instance PEG-400 or PEG-4000.

When they are present in the composition, the amount of plasticizer may represent from 0.05% to 20% by weight relative to the weight of the composition.

The composition according to the present disclosure may also comprise at least one oxidizing agent or at least one reducing agent.

Regarding the oxidizing agent, it may be chosen from hydrogen peroxide; urea peroxide; alkali metal, alkaline-earth metal or ammonium percarbonates, perborates or periodates; alkali metal, alkaline-earth metal or ammonium persulfates; alkali metal, alkaline-earth metal or ammonium bromates; ferricyanides, copper or manganese salts, and oxidizing quinones, alone or as a mixture.

In at least one embodiment, the oxidizing agent may be chosen from hydrogen peroxide and persalts.

Regarding the reducing agent, it may be chosen from reductones or sulfur-containing compounds, for example compounds containing at least one thiol, sulfite or sulfinic function, which may or may not be in salt form.

Among the reductones, non-limiting mention may be made, inter alia, of (iso)ascorbic acid, and erythorbic acid, in acid, esterified or salified form. For example, ascorbic acid or isoascorbic acid, in acid form, or a salt thereof, for instance sodium salt, is suitable for use.

Among the thiols that may be used as reducing compounds, non-limiting mention may be made of thioglycolic acid, β-mercaptoethanol, thiolactic acid, alkali metal or alkaline-earth metal salts thereof (for instance sodium, potassium or calcium salt) and esters thereof; cysteine, cysteamine and derivatives thereof; homocysteine and a salt thereof; mercaptoaldehyde; penicillamine; glutathione; glyceryl thioglycolate. These compounds may be used alone or as mixtures.

Regarding the sulfites that may be used as reducing agents, bisulfites and hydrosulfites, the alkali metal, alkaline-earth metal or ammonium salts, and also mixtures thereof, are suitable for use. For instance, mention may be made of sodium sulfite and sodium hydrosulfite.

Regarding the sulfides, among which are also included disulfides, non-limiting mention may be made of cystine.

Regarding the sulfinic compounds, non-limiting mention may be made of sodium hydroxymethanesulfinate and the compounds that are known, described and prepared in patent applications WO 99/18067 and WO 02/30369.

Reducing agents that may also be used include the sulfinic acid derivatives described in WO 03/026597 and WO 03/041668.

In at least one embodiment, the reducing agent, when it is present, is chosen from ascorbic acid, sodium hydromethanesulfinate, thiols and sodium sulfite, alone or as mixtures.

The amount of oxidizing agent in the composition, when it is present, may range from 0.1% to 40% by weight relative to the weight of the composition.

The amount of reducing agent in the composition, when it is present, may range from 0.1% to 20% by weight relative to the weight of the composition.

The composition according to the present disclosure may also comprise at least one alkaline or acidic pH regulator.

Among the alkaline agents that may be mentioned, for example, are aqueous ammonia, alkaline carbonates, alkanolamines such as monoethanolamine, diethanolamine and triethanolamine and also derivatives thereof, hydroxyalkylamines and ethylenediamines that are oxyethylenated and/or oxypropylenated, sodium hydroxide, potassium hydroxide and the compounds having the following formula: 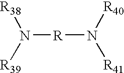
in which R is a propylene residue optionally substituted with a hydroxyl group or a C₁-C₄ alkyl radical; R₃₈, R₃₉, R₄₀ and R₄₁, which may be identical or different, represent a hydrogen atom or a C₁-C₄ alkyl or C₁-C₄ hydroxyalkyl radical.

Regarding the acidic agents, examples that may be mentioned include but are not limited to mineral or organic acids, for instance hydrochloric acid, orthophosphoric acid, sulfuric acid, carboxylic acids, for instance acetic acid, tartaric acid, citric acid and lactic acid, and sulfonic acids, or mixtures thereof.

The amount of alkaline and/or acidic agent is such that the pH of the composition, placed in contact with water, ranges from 1 to 11.

The composition as disclosed herein may also comprise conventional additives found in formulations intended for dyeing keratin fibers, provided that the composition according to the disclosure preserves the appearance of a film and that its ability to disintegrate in the presence of an aqueous medium is not substantially affected.

Examples that may be mentioned include nonionic, anionic, cationic or amphoteric surfactants; penetrants; sequestrants; fragrances; buffers; dispersants; conditioning agents, for instance cations, cationic polymers, and volatile or non-volatile, modified or unmodified silicones; ceramides; preserving agents; stabilizers; opacifiers, and the like.

The amount thereof may range from 0 to 30% by weight relative to the weight of the composition as disclosed herein.

According to at least one embodiment of the present disclosure, the composition is in the form of a film with a thickness ranging from 10 to 2000 μm such as from 20 to 500 μm.

In accordance with at least one variant of the present disclosure, the composition is deposited on a water-insoluble support that is inert with respect to the composition, and with respect to the composition when it is placed in contact with an aqueous medium.

In at least one embodiment, the water-insoluble support is chosen from polyurethanes, thermoplastic elastomers of the type such as styrene-butadiene-styrene, styrene-ethylene-butadiene-styrene, ethylene-vinyl acetate, or coether ester, polyethylenes, polypropylenes, silicones, metal sheets or films, for instance aluminium, composite sheets or films comprising polytetrafluoroethylene, polyamide copolymers containing polyether blocks, polyvinylidene chloride, nylon, elastomers of isobutylene-styrene or styrene-isoprene type, and non-woven materials.

Such supports are sold for example under the brand names Baydur®), Daltoflex®, Uroflex®, Hyperlast®, Inspire®, Desmopan®, Estane®, Lastane®, Texin®, Cariflex®, Kraton®, Solprene®, Elvax®, Escorene®, Optene®, Amitel®, Hytrel® or Riteflex®.

Alternatively, the support may be in the form of a non-woven, such as those made of cellulose, viscose, cotton or synthetic fibers.

The nature and form of the support will be chosen in an appropriate manner in order to allow the user to place the film in contact with the surface to be treated and to allow massaging on the surface without any risk to the latter and with maximum comfort.

The thickness of the support may range from 0.01 mm to 2 mm, for example from 0.02 to 0.2 mm.

The composition as disclosed herein may be obtained by applying to a support a precursor composition comprising, in a suitable solvent, a mixture comprising at least one direct dye and at least one film-forming polymer; and the said solvent is then evaporated off.

It should be noted that the amount of direct dye and of film-forming polymer in the precursor composition are such that once the solvent has evaporated off, the concentration ranges of the composition detailed previously may be found.

The precursor composition may also comprise at least one oxidizing agent or at least one pH regulator, in proportions such that the conditions described for the final composition are found.

The composition may also comprise additives that are standard in the field, and reference may be made to the list given above. In this case also, their contact in the precursor composition is such that once the solvent has evaporated off, the concentration ranges in the composition, given previously, may be found.

As disclosed herein, the precursor composition comprises at least one solvent. This solvent is chosen such that the compounds present in the precursor composition may be soluble or dispersed therein.

In at least one embodiment, the boiling point of the solvent is less than or equal to 200° C.

Examples of solvents that may be used include but are not limited to water, ethanol, acetone, isopropanol, ethyl acetate, dichloromethane, ethyl ether, etc. It should also be noted that when the precursor composition comprises both an oxidizing agent and a pH regulator chosen from alkaline compounds, then the solvent may be other than water.

The solvent amount is such that it is compatible with easy spreading the precursor composition, allowing its thickness to be controlled.

In accordance with at least one embodiment of the present disclosure, the total solvent amount is between 10% and 95% by weight relative to the weight of the precursor composition.

The composition may be obtained by mixing together the various compounds and then applying the precursor composition thus obtained to a suitable support, for instance a non-coarse, horizontal support such as marble or a heating or non-heating block.

In at least one embodiment, the composition may be directly applied onto the support with which the composition is intended to be used, if such a variant is chosen.

The composition may be deposited in a conventional manner, for instance with a device that allows a film of substantially uniform thickness to be obtained.

After depositing the composition, the solvent is evaporated off in a conventional manner, for instance in an oven.

Another aspect of the present disclosure comprises a process for dyeing keratin fibers, such as human keratin fibers, in which the fibers are placed in contact with the composition of the disclosure, in the presence of an aqueous medium.

The treated fibers may be dry or wet. In the latter case, the water present on the fibers may constitute all or part of the aqueous medium mentioned above.

Besides water, the aqueous medium may comprise one or more different adjuvants conventionally used in the field of dyeing keratin fibers.

The aqueous medium may comprise at least one oxidizing agent. This variant may be used when the composition as disclosed herein is free of oxidizing agent and when it is desired to lighten or to bleach the fibers as a function of the oxidizing agent present.

The aqueous medium may also comprise a reducing agent.

The aqueous medium may also comprise pH regulators such as basifying or acidifying agents. Reference may be made to the description as regards the nature of these agents.

Generally, the respective amounts thereof are such that the pH of the composition and of the aqueous medium ranges from 3 to 11.

The aqueous medium may also comprise adjuvants such as nonionic, anionic, cationic or amphoteric surfactants; penetrants; sequestrants; fragrances; buffers; dispersants; conditioning agents, for instance cationic polymers or volatile or non-volatile, modified or unmodified silicones; ceramides; preserving agents; stabilizers; opacifiers, and the like.

The aqueous medium may be liquid.

It is pointed out that the amount of aqueous medium may be such that it will allow the release of the dye present in the composition, via disintegration of the film.

According to at least one embodiment of the present disclosure, the composition in the form of a film according to the present disclosure and the aqueous medium are successively applied, or alternatively the composition according to the action is applied to the wet fibers, optionally followed by application of an additional aqueous medium. Once these operations have been performed, the fibers may be massaged in order to promote the disintegration of the film and the distribution of the dye thus released onto all of the fibers to be treated.

According to at least one embodiment, prior to the application onto the wet or dry keratin fibers, mixing of the composition according to the present disclosure with the aqueous medium is performed.

Irrespective of the variant adopted, the composition is left to stand on the fibers until the desired coloration has been obtained.

Purely as a guide, the leave-in time may generally range from 1 to 60 minutes, for example from 5 to 45 minutes.

The application temperature usually ranges from 15° C. to 220° C. and in at least one embodiment, the application temperature may be in the region of room temperature.

Once the leave-in time has passed, the fibers are rinsed, optionally washed with a shampoo and then rinsed again, before being dried or left to dry.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contains certain errors necessarily resulting from the standard deviation found in their respective testing measurements.

The embodiments disclosed herein are illustrated in greater detail by the non-limiting examples described below.

### EXAMPLE 1

| | |
|---|---|
| Hydroxypropylmethylcellulose | 10 g |
| Basic Red 51 | 15 g |
| Water | 42.5 g |

The ingredients above were all mixed together with stirring.

The solution was deposited on a silicone paper to a thickness of about 850 μm, and was then dried at a temperature of 50° C.

After drying, the film obtained was cut into strips 40 mm wide and 80 mm long.

100 g of water were spread on a head, then immediately after, two strips obtained above were applied and were moved over the head of hair by massaging.

The hair was left for 30 minutes.

After rinsing, shampooing and drying, the head of hair had a strong red tint.

### EXAMPLE 2

| | |
|---|---|
| Hydroxypropylcellulose | 10 g |
| Basic Red 51 | 5 g |
| Potassium persulfate | 25 g |
| Ethanol | 32 g |

The ingredients were mixed together with stirring.

The dispersion was deposited on a silicone paper to a thickness of about 1 mm and was then dried at room temperature.

After drying, the film obtained was cut into strips 40 mm wide and 80 mm long.

2 g of a bleaching cream, obtained by mixing 0.8 g of an anhydrous composition A described below with 1.2 g of an aqueous hydrogen peroxide composition at 6% by weight, were applied to a lock of dark chestnut-brown hair.

Composition A

| | | |
|---|---|---|
| Sodium disilicate | 15.0 | g |
| Sodium metasilicate | 3.0 | g |
| Ammonium chloride | 4.2 | g |
| EDTA | 0.2 | g |
| Serad FX 1100 | 2.0 | g |
| Primogel | 2.0 | g |
| Sodium lauryl sulfate | 4 | g |
| Sodium stearate | 2 | g |
| Magnesium stearate | 2 | g |
| Amorphous silica | 1 | g |
| Titanium oxide | 1 | g |
| Mineral oil | 1 | g |
| Beeswax | 1 | g |
| Isopropyl myristate | 21.6 | g |

Immediately after applying the resulting mixture, two strips obtained above were applied to the lock and were then moved over the lock by massaging.

The lock was left to stand for 40 minutes.

After rinsing and drying, a blond lock with auburn tints is obtained.

What is claimed is:

1. An anhydrous composition in the form of a film comprising
at least one film-forming polymer and
at least one direct dye,
wherein the at least one direct dye is present in an amount of at least 3% by weight relative to the total weight of the composition.

2. The composition according to claim 1, wherein the at least one direct dye is chosen from nitrobenzene dyes, azo dyes, azomethine dyes, methine dyes, tetraazapentamethine dyes, anthraquinone dyes, naphthoquinone dyes, benzoquinone dyes, phenothiazine dyes, indigoid dyes, xanthene dyes, phenanthridine dyes, phthalocyanin dyes, triarylmethane-based dyes, natural dyes, and mixtures thereof.

3. The composition according to claim 1, wherein the at least one direct dye is present in an amount ranging from 3% to 99.5% by weight relative to the total weight of the composition.

4. The composition according to claim 3, wherein the at least one direct dye is present in an amount ranging from 10% to 80% by weight relative to the total weight of the composition.

5. The composition according to claim 1, wherein the at least one film-forming polymer is chosen from water-soluble and water-dispersible film-forming polymers.

6. The composition according to claim 5, wherein the at least one film-forming polymer is a water-soluble film-forming polymer.

7. The composition according to claim 1, wherein the at least one film-forming polymer is present in the composition in an amount ranging from 0.5% to 97% by weight relative to the total weight of the composition.

8. The composition according to claim 1, further comprising at least one plasticizer.

9. The composition according to claim 8, wherein the at least one plasticizer is chosen from glycerol, sorbitol, monosaccharides and/or disaccharides, dipropylene glycol, butylene glycol, pentylene glycol and polyethylene glycol.

10. The composition according to claim 8, wherein the plasticizer is present in the composition in an amount ranging from 0.05% to 20% by weight relative to the total weight of the composition.

11. The composition according to claim 1, wherein the film has a thickness ranging from 10 to 2000 μm

12. The composition according to claim 11, wherein the film has a thickness ranging from 20 to 500 μm.

13. The composition according to claim 1, wherein said composition comprises water in an amount of less than 10% by weight relative to the total weight of the composition.

14. The composition according to claim 13, wherein the composition comprises water in an amount of less than 3% by weight relative to the total weight of the composition.

15. The composition according to claim 1, further comprising at least one oxidizing agent.

16. The composition according to claim 15, wherein the at least one oxidizing agent is chosen from hydrogen peroxide; urea peroxide; alkali metal, alkaline-earth metal or ammonium percarbonates, perborates or periodates; alkali metal, alkaline-earth metal or ammonium persulfates; alkali metal, alkaline-earth metal or ammonium bromates; ferricyanides, copper or manganese salts, oxidizing quinones, and mixtures thereof.

17. The composition according to claim 1, wherein the composition further comprises at least one reducing agent.

18. The composition according to claim 17, wherein the at least one reducing agent is chosen from reductones and sulfur-containing compounds.

19. The composition according to claim 18, wherein said compounds are chosen from compounds comprising at least one function chosen from thiol, sulfite and sulfinic functions, optionally in salt form.

20. The composition according to claim 1, wherein the composition further comprises at least one pH regulator.

21. The composition according to claim 1, wherein said composition is deposited onto a water-insoluble support.

22. The composition of claim 21, wherein the water-insoluble support is chosen from polyurethanes, thermoplastic elastomers, polyethylenes, polypropylenes, silicones, metal sheets or films, composite sheets or films comprising polytetrafluoroethylene, polyamide copolymers comprising polyether blocks, polyvinylidene chloride, nylon, elastomers of the isobutylene-styrene or styrene-isoprene type, and non-woven materials.

23. The composition of claim 22, wherein the thermoplastic elastomers are chosen from styrene-butadiene-styrene, styrene-ethylene-butadiene-styrene, ethylene-vinyl acetate, and coether ester.

24. The composition of claim 22, wherein the metal sheets or films are chosen from aluminum.

25. The composition of claim 22, wherein the non-woven materials comprise fibers chosen from cellulose, viscose, cotton, and synthetic fibers.

26. A process for preparing an anhydrous composition in the form of a film comprising at least one film-forming polymer and at least 3% by weight of at least one direct dye relative to the total weight of the composition, said process comprising
applying to a support a precursor composition comprising, in a suitable solvent, a mixture comprising at least one direct dye and at least one film-forming polymer; and
then evaporating off said solvent.

27. The process according to claim 26, wherein the precursor composition comprises a total solvent amount ranging from 10% to 95% by weight relative to the total weight of the composition.

28. A process for dyeing keratin fibers, comprising
placing said fibers in contact with an anhydrous composition in the form of a film comprising at least one film-forming polymer and at least one direct dye, wherein the at least one direct dye is present in an amount of at least 3% by weight relative to the total weight of the composition, in the presence of an aqueous medium.

29. The process according to claim 28, wherein the aqueous medium comprises at least one oxidizing agent.

30. The process according to claim 28, wherein the aqueous medium comprises at least one reducing agent.

31. The process according to claim 26, wherein the composition and the aqueous medium are applied successively.

32. The process according to claim 26, wherein the composition and the aqueous medium are applied together as a mixture.
